(19) United States
(12) Patent Application Publication
(10) Pub. No.: US 2007/0179175 A1
(43) Pub. Date: Aug. 2, 2007

(54) Tetrahydronaphthyridine Derivative

(76) Inventor: Graham Lunn, Sandwich (GB)

Correspondence Address: PHARMACIA CORPORATION;GLOBAL PATENT DEPARTMENT, POST OFFICE BOX 1027, ST. LOUIS, MO, 63006, US

(21) Appl. No.: 11/555,927

(22) Filed: Nov. 2, 2006

Related U.S. Application Data
(60) Provisional application No. 60/733,590, filed on Nov. 4, 2005.

(51) Int. Cl. A61K 31/4745; C07D 471/02

(52) U.S. Cl. 514/300; 546/122

(57) ABSTRACT

The invention relates to 6-[2-(1-Isopropyl-piperidin-4-yloxy)-7,8-dihydro-5H-[1,6]naphthyridin-6-yl]-nicotinamide and to processes for the preparation of, compositions containing and the uses of, the compound. The compound is an H3 ligand and is useful in numerous diseases, disorders and conditions, in particular inflammatory, allergic and respiratory diseases, disorders and conditions.

### CROSS REFERENCE TO RELATED APPLICATIONS

Priority is claimed to U.S. Provisional Application Ser. No. 60/733,590, filed 4 Nov. 2005, which is hereby incorporated by reference.

### BACKGROUND OF THE INVENTION

Histamine H₃ receptors are found inter alia on presynaptic terminals if peripheral nerves, where they modulate autonomic neurotransmission and modulate a variety of end organ responses under control of the autonomic nervous system. They are also heteroreceptors, modulating the release of numerous other neurotransmitters such as dopamine, glutamate, noradrenaline, serotonin, GABA, acetylcholine, some peptides and co-transmitters.

Recently, numerous histamine H₃ receptor ligands have been developed. An overview of the current advance in H₃ ligand research and patenting is given in Expert Opin. Ther. Patents (2003) 13(6). Examples of Histamine H₃ receptor ligands can be found in WO02/76925, WO00/06254, WO02/12190, WO02/12214 and WO02/06223.

H₃ receptor ligands are believed to be suitable for the treatment of various diseases including both disorders of the central nervous system and inflammatory disorders. Examples of diseases where treatment with H₃ ligands is believed to be useful are inflammatory bowel disease, Crohn's disease, colitis ulcerosa, sleep disorders, migrane, dyskinesia, stress-induced anxiety, psychotic disorders, epilepsy, Cognition deficiency diseases such as Alzheimer's disease or mild cognitive impairment, depression, mood disorders, schizophrenia, anxiety disorders, attention-deficit hyperactivity disorder (ADHD), psychotic disorders, obesity, dizziness, epilepsy, motion sickness, vertigo, female and male sexual dysfunction, respiratory diseases such as adult respiratory distress syndrome, acute repiratory distress syndrome, bronchitis, chronic bronchitis, chronic obstructive pulmonary disease, cystic fibrosis, asthma, emphysema, rhinitis, chronic sinusitis, allergy, allergy-induced airway responses, allergic rhinitis, viral rhinitis, non-allergic rhinitis, perennial and seasonal rhinitis, nasal congestion and allergic congestion.

Although H₃ ligands are known, there is still a need to provide new H₃ ligands that are good drug candidates. In particular, preferred compounds should bind potently to the histamine H₃ receptor whilst showing little affinity for other receptors. They should be well absorbed from the gastrointestinal tract, be metabolically stable and possess favorable pharmacokinetic properties. They should be non-toxic and demonstrate few side-effects.

### SUMMARY OF THE INVENTION

The present invention therefore provides a compound which is 6-[2-(1-isopropyl-piperidin-4-yloxy)-7,8-dihydro-5H-[1,6]naphthyridin-6-yl]-nicotinamide 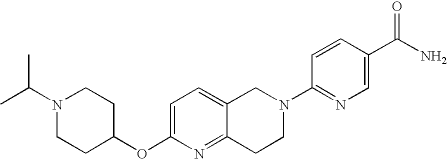
and pharmaceutically and/or veterinarily acceptable derivatives thereof.

In particular, the invention provides the above compound other than when formed in vivo.

This compound may combine an increased H₃ potency with a potential for reduced cardiovascular side effects. Assays for determining H₃ potency and cardiovascular side effects are given in the experimental section hereafter (H₃ cell based functional assay and a hERG product based functional assay, respectively). This compound may also have the advantage that it is more potent, has a longer duration of action, has a broader range of activity, is more stable, has fewer side effects or is more selective, or has other more useful properties than the compounds of the prior art.

By pharmaceutically and/or veterinarily acceptable derivative it is meant any pharmaceutically or veterinarily acceptable salt, solvate, ester or amide, or salt or solvate of such ester or amide, of the compound or any other compound which upon administration to the recipient is capable of providing (directly or indirectly) the compound or an active metabolite or residue thereof.

Pharmaceutically acceptable salts of the compound include the acid addition salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Hemisalts of acids may also be formed, for example, hemisulphate salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts of the compound may be prepared by one or more of three methods:
(i) by reacting the compound with the desired acid;
(ii) by removing an acid- or base-labile protecting group from a suitable precursor of the compound or by ring-opening a suitable cyclic precursor, for example, a lactone or lactam, using the desired acid or base; or
(iii) by converting one salt of the compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compound of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter all references to the compound of the invention include references to salts, solvates and complexes thereof and to solvates and complexes of salts thereof.

The compound of the invention includes all polymorphs and crystal forms thereof, prodrugs and isomers thereof (including optical, geometric and tautomeric isomers) as hereinafter defined and isotopically-labeled compounds.

As indicated, so-called ‘pro-drugs’ of the compound are also within the scope of the invention. Thus certain derivatives of the compound which may have little or no pharmacological activity themselves can, when administered into or onto the body, be converted into the compound, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of the invention with certain moieties known to those skilled in the art as ‘pro-moieties’ as described, for example, in Design of Prodrugs by H. Bundgaard (Elsevier, 1985).

Included within the scope of the present invention are all stereoisomers, geometric isomers and tautomeric forms of the compound, including compounds exhibiting more than one type of isomerism, and mixtures of one or more thereof. Also included are acid addition salts wherein the counterion is optically active, for example, d-lactate or l-lysine, or racemic, for example, dl-tartrate or dl-arginine.

Stereoisomeric conglomerates may be separated by conventional techniques known to those skilled in the art—see, for example, Stereochemistry of Organic Compounds by E. L. Eliel and S. H. Wilen (Wiley, New York, 1994).

The present invention includes all pharmaceutically acceptable isotopically-labelled derivatives of the compound wherein one or more atoms are replaced by atoms having the same atomic number, but an atomic mass or mass number different from the atomic mass or mass number which predominates in nature.

Examples of isotopes suitable for inclusion in the compound of the invention include isotopes of hydrogen, such as ²H and ³H, carbon, such as ¹¹C, ¹³C and ¹⁴C, nitrogen, such as ¹³N and ¹⁵N, and oxygen, such as ¹⁵O, ¹⁷O and ¹⁸O.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁵O and ¹³N, can be useful in Positron Emission Topography (PET) studies for examining substrate receptor occupancy.

Isotopically-labeled compounds of the invention can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₅-acetone, d₆-DMSO.

The compound according to the present invention can be prepared by the specific methods described in the Examples section and the Preparations section set out below, or by routine modifications thereof.

The compound of the invention intended for pharmaceutical use may be administered as crystalline or amorphous products. It may be obtained, for example, as a solid plug, a powder, or a film by methods such as precipitation, crystallization, freeze-drying, spray drying, or evaporative drying. Microwave or radio frequency drying may be used for this purpose.

It may be administered alone or in combination with one or more other active drugs. Generally, they will be administered as a formulation in association with one or more pharmaceutically acceptable excipients. The term ‘excipient’ is used herein to describe any ingredient other than the compound of the invention. The choice of excipient will to a large extent depend on factors such as the particular mode of administration, the effect of the excipient on solubility and stability, and the nature of the dosage form.

Pharmaceutical compositions suitable for the delivery of the compound of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compound of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compound of the invention may also be used in fast-dissolving, fast-disintegrating dosage forms such as those described in Expert Opinion in Therapeutic Patents, 11 (6), 981-986, by Liang and Chen (2001). For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise the compound of the invention, a film-forming polymer, a binder, a solvent, a humectant, a plasticiser, a stabiliser or emulsifier, a viscosity-modifying agent and a solvent. Some components of the formulation may perform more than one function.

The compound of the invention may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of the invention may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compound of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the compound of the invention used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus the compound of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compound of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compound of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container (e.g. a metered dose inhaler), pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise the compound of the invention, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount or the drug product is packaged as discrete single dose units for use in the inhaler device. The inhaler devices are typically arranged to administer a metered dose or “puff” containing from 1 μg to 4000 μg of the compound of the invention. The overall daily dose will typically be in the range 1 μg to 20 mg which may be administered in a single dose or, more usually, as divided doses throughout the day.

The compound of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compound of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compound of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains the compound in accordance with the invention, may conveniently be combined in the form of a kit suitable for coadministration of the compositions.

Thus the kit of the invention comprises two or more separate pharmaceutical compositions, at least one of which contains the compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compound of the invention is typically in the 0.001 mg to 2000 mg depending, of course, on the mode of administration. For example, oral administration may require a total daily dose of from 1 mg to 2000 mg, while an intravenous dose may only require from 0.01 mg to 100 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

According to another embodiment of the present invention, the compound of the invention, or pharmaceutically acceptable salts, derived forms or compositions thereof, can also be used as a combination with one or more additional therapeutic agents to be co-administered to a patient to obtain some particularly desired therapeutic end result. The second and more additional therapeutic agents may also be one or more histamine H₃ receptor ligands known in the art. More typically, the second and more therapeutic agents will be selected from a different class of therapeutic agents.

As used herein, the terms “co-administration”, “co-administered” and “in combination with”, referring to the compound of the invention and one or more other therapeutic agents, is intended to mean, and does refer to and include the following:
simultaneous administration of such combination of the compound of the invention and one or more therapeutic agent(s) to a patient in need of treatment, when such components are formulated together into a single dosage form which releases the components at substantially the same time to the patient,
substantially simultaneous administration of such combination of the compound of the invention and one or more therapeutic agent(s) to a patient in need of treatment, when such components are formulated apart from each other into separate dosage forms which are taken at substantially the same time by the patient, whereupon the components are released at substantially the same time to the patient,
sequential administration of such combination of the compound of the invention and one or more therapeutic agent(s) to a patient in need of treatment, when such components are formulated apart from each other into separate dosage forms which are taken at consecutive times by the patient with a significant time interval between each administration, whereupon said components are released at substantially different times to the patient; and
sequential administration of such combination of the compound of the invention and one or more therapeutic agent(s) to a patient in need of treatment, when such components are formulated together into a single dosage form which releases the components in a controlled manner whereupon they are concurrently, consecutively, and/or overlapingly administered at the same and/or different times by or to the patient,

where each part may be administered by either the same or different route.

Suitable examples of other therapeutic agents which may be used in combination with the compound of the invention, or pharmaceutically acceptable salts, derived forms or compositions thereof, include, but are by no means limited to:
Histamine H₁ receptor antagonists, for instance loratidine, desloratidine, fexofenadine and cetirizine,
Histamine H₄ receptor antagonists,
Histamine H₂ receptor antagonists,
Leukotriene antagonists, including antagonists of LTB₄, LTC₄, LTD₄, and LTE₄, in particular Montelukast,
Phosphodiesterase inhibitors such as PDE4 inhibitors or PDE5 inhibitors,
neurotransmitter re-uptake inhibitors, for instance fluoxetine, setraline, paroxetine, ziprasidone,
5-Lipoxygenase (5-LO) inhibitors or 5-lipoxygenase activating protein (FLAP) antagonists,
α₁- and α₂-adrenoceptor agonist vasoconstrictor sympathomimetic agents for decongestant use,
Muscarinic M3 receptor antagonists or anticholinergic agents,
β₂-adrenoceptor agonists,
Theophylline,
Sodium cromoglycate,
COX-1 inhibitors (NSAIDs) and COX-2 selective inhibitors,
Oral or inhaled Glucocorticosteroids,
Monoclonal antibodies active against endogenous inflammatory entities,
Anti-tumor necrosis factor (anti-TNF-α) agents,
Adhesion molecule inhibitors including VLA-4 antagonists,
Kinin-B₁- and B₂-receptor antagonists,
Immunosuppressive agents,
Inhibitors of matrix metalloproteases (MMPs),
Tachykinin NK₁, NK₂ and NK₃ receptor antagonists,
Elastase inhibitors,
Adenosine A2a receptor agonists,
Inhibitors of urokinase,
Compounds that act on dopamine receptors, e.g. D2 agonists,
Modulators of the NFκβ pathway, e.g. IKK inhibitors,
Agents that can be classed as mucolytics or anti-tussive,
antibiotics,
modulators of cytokine signalling pathyways such as p38 MAP kinase, syk kinase or JAK kinase inhibitor,
HDAC inhibitors, and
P13 kinase inhibitors.

According to the present invention, combination of the compound of the invention with Histamine H1 receptor antagonists (e.g. loratidine, desloratidine, fexofenadine and cetirizine), Histamine H₄ receptor antagonists, Histamine H₂ receptor antagonists, Leukotriene antagonists, including antagonists of LTB₄, LTC₄, LTD₄, and LTE₄ (in particular Montelukast), Phosphodiesterase PDE4 inhibitors and neurotransmitter re-uptake inhibitors (e.g. fluoxetine, setraline, paroxetine, duloxetine, ziprasidone) are preferred.

The compound of the invention has the ability to interact with the H₃ receptor and thereby has a wide range of therapeutic applications, as described further below, because of the essential role which the H₃ receptor plays in the physiology of all mammals. According to this invention H₃ ligands are meant to include H₃ receptor antagonists, agonists and inverse agonists. For the preferred indications to be treated according to the invention, H₃ antagonists are believed to be most suitable.

Therefore, a further aspect of the present invention relates to the compound of the invention, or pharmaceutically acceptable salts, derived forms or compositions thereof, for use in the treatment of diseases, disorders, and conditions in which the H₃ receptor is involved. More specifically, the present invention also concerns the compound of the invention, or pharmaceutically acceptable salts, derived forms or compositions thereof, for use in the treatment of diseases, disorders, and conditions selected from the group consisting of:
diseases of the central nervous system: sleep disorders, migraine, dyskinesia, stress-induced anxiety, psychotic disorders, epilepsy, Cognition deficiency diseases such as Alzheimer's disease or mild cognitive impairment, depression, mood disorders, schizophrenia, anxiety disorders, attention-deficit hyperactivity disorder (ADHD), psychotic disorders, dizziness, vertigo, epilepsy, motion sickness
eating disorders (in particular, obesity-related eating disorders), weight loss or control (e.g., reduction in calorie or food intake, and/or appetite suppression), and obesity. Representative examples of obesity-related eating disorders include overeating, bulimia, binge-eating disorder, compulsive dieting, nocturnal sleep-related eating disorder, pica, Prader-Willi Syndrome, and night-eating syndrome
inflammatory diseases
respiratory diseases (adult respiratory distress syndrome, acute respiratory distress syndrome, bronchitis, chronic bronchitis, chronic obstructive pulmonary disease, cystic fibrosis, asthma, emphysema, rhinitis, chronic sinusitis), allergy, allergy-induced airway responses, allergic rhinitis, viral rhinitis, non-allergic rhinitis, perennial and seasonal rhinitis, nasal congestion, allergic congestion
Female sexual dysfunction including hypoactive sexual desire disorder, sexual arousal disorder, orgasmic disorder and sexual pain disorder
Male sexual dysfunction including male desire disorders, male erectile dysfunction, male orgasmic disorders such as premature ejaculation
cardiac dysfunctions such as myocardial ischaemia and arrythmia
diseases of the gastrointestinal tract such as inflammatory bowel disease, Crohn's disease and colitis ulcerosa
cancer
hypotension
pain and
overactive bladder conditions

The compound of the invention is particularly suitable for the treatment of allergy, allergy-induced airway responses, allergic rhinitis, viral rhinitis, non-allergic rhinitis, perennial and seasonal rhinitis, nasal congestion and allergic congestion.

A still further aspect of the present invention also relates to the use of the compound of the invention, or pharmaceutically acceptable salts, derived forms or compositions thereof, in the manufacture of a drug being a H₃ ligand. In particular, the present inventions concerns the use of the compounds of the invention, or pharmaceutically acceptable salts, derived forms or compositions thereof, in the manufacture of a drug for the treatment of H3-mediated diseases and/or conditions, in particular the diseases and/or conditions listed above.

As a consequence, the present invention provides a particularly interesting method to treat a mammal, including a human being, with an effective amount of the compound of the invention, or a pharmaceutically acceptable salt, derived form or composition thereof. More precisely, the present invention provides a particularly interesting method for the treatment of a H3-mediated diseases and/or conditions in a mammal, including a human being, in particular the diseases and/or conditions listed above, comprising administering to said mammal an effective amount of the compound of the invention, its pharmaceutically acceptable salts and/or derived forms.

The following example illustrate the preparation of the compound of the invention.

### EXAMPLE

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used: ‘Ammonia’ refers to a concentrated solution of ammonia in water possessing a specific gravity of 0.88. Where thin layer chromatography (TLC) has been used it refers to silica gel TLC using silica gel 60 F₂₅₄ plates, R_{f} is the distance travelled by a compound divided by the distance travelled by the solvent front on a TLC plate.

Preparation 1

### 6-Benzyl-5,6,7,8-tetrahydro-1,6-naphthyridin-2(1H)-one

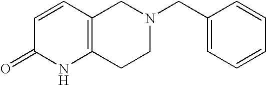

To diethylene glycol (200 ml) at 230° C. was added over 2 minutes 6-benzyl-5,6,7,8-tetrahydro-2(1H)-oxo-1,6-napthyridine-3-carboxylic acid hydrochloride [(40 g, 124.8 mmol), CA 2104267], and mixture continued stirring at 230° C. for further 8 minutes. The mixture was poured onto ice (1 kg) and an excess of solid sodium hydrogen carbonate was added to basify. The mixture was filtered, then extracted with dichloromethane (2×400ml). The combined organic phase was washed with water (2×400 ml), dried over sodium sulfate and concentrated in vacuo. The residue was triturated with ethyl acetate (250 ml), then filtered and dried in vacuo to afford the title compound as an off white solid in 46% yield, 14 g. ¹HNMR(DMSO-D₆, 400 MHz) δ: 2.53(m, 2H), 2.61(m, 2H), 3.20(s, 2H), 3.60(s, 2H), 6.08(d, 1H), 7.10(d, 1 H), 7.24(m,1 H), 7.32(m, 4H), 11.40(brs,1H)

MS APCI+m/z 241 [MH]⁺

### Preparation 2

### 6-Benzyl-2-chloro-5,6,7,8-tetrahydro-1,6-naphthyridine

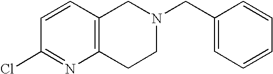

A mixture of the 6-benzyl-5,6,7,8-tetrahydro-1,6-naphthyridin-2(1H)-one (preparation 1, 50 g, 208 mmol), phosphorous oxychloride (250 ml) and tetraethylammonium chloride hydrate (35 g, 210 mmol) was heated under reflux for 2 hours 30 minutes. The phosphorous oxychloride was removed by distillation, then the residue cooled and diluted with dichloromethane (1000 ml). The mixture was added over 10 minutes to a mixture of sodium hydrogen carbonate (240 g), water (1000 ml), and dichloromethane (600 ml), and stirred for 75 minutes. The organic layer was separated, washed further with water, then concentrated in vacuo. The residue was dissolved in ethyl acetate (700 ml), and washed with water (100 ml) then saturated aqueous sodium hydrogen sulfate solution (50 ml). The organic phase was separated then purified by column chromatography on silica gel, eluting with ethyl acetate, to afford the title product as a white solid in 81% yield, 43.6 g.

¹HNMR(DMSO-D₆, 400 MHz) δ: 2.81(m, 2H), 3.05(m, 2H), 3.60(s, 2H), 3.74(s, 2H), 7.05(d, 1H), 7.20-7.40(m, 6H)

MS APCl⁺ m/z 259 [MH]⁺

Alternatively the title compound can be prepared by the following method:

To a mixture of 1-benzyl-4-piperidone (40 g, 210 mmol) and acetamide (40 g, 678 mmol) in toluene (200 ml) stirred at 50° C. was added over 1 minute p-toluenesulfonic acid monohydrate (43 g, 226 mmol), then the mixture was heated under reflux, with the removal of water under Dean and Stark conditions, for 3 hours. The mixture was cooled then concentrated in vacuo. The residue was partitioned between dichloromethane (300 ml) and a solution of sodium hydrogen carbonate (20 g) in water (1000 ml). Further sodium hydrogen carbonate (60 g) was added until effervescence ceased. The organic later was separated, dried over sodium sulfate and concentrated in vacuo. The residue was triturated with ether (200 ml) and dried in vacuo to give N-(1-benzyl-1,2,3,6-tetrahydro-pyridin-4-yl)-acetamide as an orange solid in 50% yield, 24.4g.

¹HNMR(CDCl₃, 400 MHz) δ: 2.02(s, 3H), 2.29(m, 2H), 2.62(m, 2H), 3.06(m, 2H), 3.58(s, 2H), 6.09(s, 1H), 6.36(s, 1H), 7.26-7.35(m, 5H).

Dimethyl formamide (7.9 ml, 102 mmol), was added to phosphorous oxychloride (250 ml) with ice cooling, then allowed to warm to room temperature and stirred for one hour. The mixture was cooled again in an ice bath and N-(1-benzyl-1,2,3,6-tetrahydro-pyridin-4-yl)-acetamide (23.4 g, 101 mmol) was added, and stirred with cooling for 10 minutes, then the mixture was allowed to warm to room temperature and was stirred for 16 hours. The mixture was concentrated in vacuo, and azeotroped with toluene (150 ml). The residue was dissolved in dichloromethane (400 ml) and ice water (100 ml) was added. To the mixture was added 0.880 ammonia solution and further ice until pH>10 and no further precipitate formed. Further dichloromethane (50 ml) was added. The organic extract was separated, dried over sodium sulfate and concentrated in vacuo. The residue was purified by column chromatography on silica gel, eluting with dichloromethane:ethyl acetate, 1:1. Appropriate fractions were concentrated in vacuo. The residue was dissolved in diethyl ether (200 ml) and washed with a solution of sodium hydrogensulfite (800 mg) in water (50 ml), then further with water (20 ml). The organic layer was dried over sodium sulfate and concentrated in vacuo. The residue was triturated with diisopropyl ether (20 ml), then dried in vacuo to afford the title product as a pale yellow solid in 21% yield, 5.54 g.

### Preparation 3

### 1-Isopropyl-piperidin-4-ol

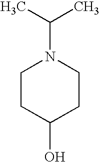

A mixture of 4-hydroxypiperidine (10 g, 0.10 mol), acetone (21.8 ml, 0.30 mol), acetic acid (5.7 ml, 0.10 mol) and tetrahydrofuran (150 ml) was stirred in an ice bath for 15 minutes. Sodium triacetoxyborohydride (31.3 g, 0.15 mol) was then added portionwise and the mixture was stirred for a further 10 minutes. The reaction mixture was then warmed and stirred at room temperature for 10 minutes and at 40° C. for 2.5 hours. The solvent was evaporated under reduced pressure and the residue was dissolved in water (50 ml). The aqueous solution was basified to pH9 with 0.88 ammonia and the solution was stirred for 30 minutes. The reaction mixture was then extracted with diethyl ether (2×200 ml) and the combined extracts were dried over sodium sulfate and concentrated in vacuo to give a yellow oil. The oil was purified by column chromatography on silica gel, eluting with dichloromethane:methanol:0.88 ammonia, 96:4:1 to 90:10:1, to afford the title product as a yellow oil in quantitative yield, 14.6 g.

¹HNMR(CDCl₃, 400 MHz) δ: 0.92-1.02(m, 6H), 1.41-1.57(m, 2H), 1.77-1.89(m, 2H), 2.07-2.23(m, 2H), 2.57-2.78(m, 3H), 3.43-3.85(brm, 2H)

MS ES+ m/z 144 [MH]⁺

### Preparation 4

### 6-Benzyl-2-[(1-isopropylpiperidin-4-yl)oxy]-5,6,7,8-tetrahydro-1,6-naphthyridine

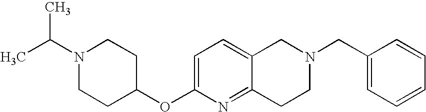

A solution of potassium tert-butoxide (9.3 g, 83 mmol) in tetrahydrofuran (100 ml), was added, with ice cooling under nitrogen over 10 minutes, to a solution of 1-isopropyl-piperidin-4-ol [(preparation 3) 12 g, 84 mmol] in tetrahydrofuran (100 ml) and the solution was stirred with warming to 13° C. over 15 minutes. To the mixture was added 6-benzyl-2-chloro-5,6,7,8-tetrahydro-1,6-naphthyridine [preparation 2 (17 g, 60 mmol)] then mixture was heated under reflux for 28 hours. The reaction mixture was then cooled to room temperature and evaporated under reduced pressure. The residue was partitioned between diethyl ether (400 ml) and water (100 ml). The organic layer was washed further with water (2×100 ml) then brine (50 ml). The organic layer was concentrated in vacuo then redissolved in ethylacetate (400 ml) and washed with water (2×100 ml) then brine (50 ml). The organic layer was dried over sodium sulfate, and concentrated in vacuo to give the title compound impure as a pale orange solid in 90% yield (by NMR), 24 g. Further purification could be achieved by column chromatography on silica gel, eluting with dichloromethane:methanol: 0.88 ammonia, 96:4:1 to 95:5:1, to afford the title compound as a white solid. ¹HNMR(CD₃OD, 400 MHz) δ: 1.02-1.15(m, 6H), 1.71-1.86(m, 2H), 1.97-2.10(m, 2H), 2.39-2.57(m, 2H), 2.71-2.94(m, 7H), 3.54(s, 2H), 3.65-3.75(m, 2H), 4.93-5.05(m, 1H), 6.52(d, 1H), 7.21-7.45(m, 6H)

MS APCl+ m/z 366 [MH]⁺

### Preparation 5

### 2-[(1-Isopropylpiperidin-4-yl)oxy]-5,6,7,8-tetrahydro-1,6-naphthyridine

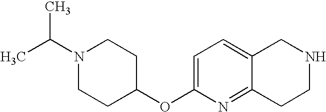

Palladium (II) hydroxide 20% on carbon [Pearlman's Catalyst, (6 g)] was added to a solution of 6-benzyl-2-[(1-isopropylpiperidin-4-yl)oxy]-5,6,7,8-tetrahydro-1,6-naphthyridine [preparation 4 (57.7g, 157 mmol)] and 2M hydrochloric acid (200 ml) in ethanol (300 ml) and the mixture was stirred under 50 psi of hydrogen for 4 hours at 50° C. The mixture was then filtered through Arbocel®, washing through with ethanol (200 ml), and the filtrate was concentrated in vacuo. To the residue was added water (200 ml) and an excess of 0.880 ammonia solution to basify, and the mixture was extracted with ethyl acetate (4×250 ml). The organic phase was dried over sodium sulfate, and concentrated in vacuo to give the title product as a colourless oil in 83% yield, 36.4 g.

¹HNMR(CDCl₃, 400 MHz) δ: 1.01-1.15(m, 6H), 1.74-1.91(m, 2H), 2.02-2.19(m, 2H), 2.40-2.58(m, 2H), 2.69-2.88(m, 5H), 3.07-3.22(m, 2H), 3.80-3.91 (m, 2H), 4.95-5.10(m, 1H), 6.44(d, 1H), 7.14(m, 1H)

MS APCl+ m/z 276 [MH]⁺

### Preparation 6

### 6-Bromo-nicotinamide

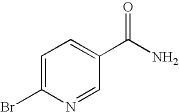

To a solution of 6-bromo-nicotinic acid (4.8 g, 23.8 mmol) in dimethylsulfoxide (20 ml) was added at room temperature carbonyldiimidazole (4.8 g, 29.6 mmol), and the mixture stirred for 16 hours. To the mixture was added dropwise, with cooling in ice bath, 0.880 ammonia solution (40 ml), then the mixture stirred for 1 hour, then poured into water (20 ml). The precipitate was filtered, washed with water and dried in vacuo to give the title product as a white solid in 81% yield, 3.9 g. ¹HNMR(DMSO-D₆, 300 MHz) δ: 7.66(br.s, 1H), 7.73(d, 1H), 8.09(m, 1H), 8.15(br.s, 1H), 8.78(d, 1H). micro analysis found (%); C(36.00), H(2.60), N(13.67); C₆H₅N₂Br requires (%); C(35.84), H(2.51), N(13.93)

### Example 1

### 6-[2-(1-Isopropyl-piperidin-4-yloxy)-7,8-dihydro-5H-[1,6]naphthyridin-6-yl]-nicotinamide

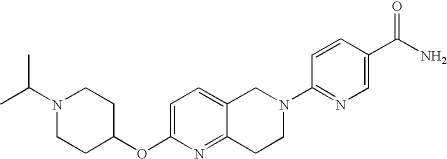

To a solution of 2-[(1-isopropylpiperidin-4-yl)oxy]-5,6,7,8-tetrahydro-1,6-naphthyridine [preparation 5, (275 mg, 1.0 mmol) and diisopropyl amine (0.345 ml, 2.0 mmol) in 2-methyl-2-butanol (3 ml) was added 6-bromo-nicotinamde [preparation 6, (200 mg, 1.0 mmol), and the mixture heated for 10 hours. The mixture was cooled, and resulting precipitate filftered and washed further with 2-methyl-2-butanol (4 ml). The solid was then partitioned between ethylacetate (50 ml) and 1 N sodium hydroxide solution (40 ml), and a drop of methanol. The organic layer was washed with brine (2×10 ml), dried over magnesium sulfate, and concentrated in vacuo. The residue was recrystallised from refluxing ethyl acetate (10 ml), with hot filtration. After cooling the resulting solid was filtered and dried in vacuo to afford the title compound as a white solid in 27% yield, 108 mg.

¹HNMR(DMSO-D₆, 400 MHz) δ: 0.92(d, 6H), 1.54(m, 2H), 1.89(m, 2H), 2.24(m, 2H), 2.60-2.70(m, 3H), 2.79(m, 2H), 3.91(m, 2H), 4.63(s, 2H), 4.91(m, 1H), 6.57(d, 1H), 6.86(d, 1H), 7.10(br.s, 1H), 7.50(d, 1H), 7.73(br.s, 1H), 7.93(m, 1H), 8.59(m, 1H)

MS APCl⁺ m/z 396 [MH]⁺

### hERG Patch Clamp Assay

To determine the potential of compounds to inhibit the hERG channel, the cloned counterpart of the rapidly inactivating delayed rectifier potassium current (IKr), HEK293 cells stably expressing the hERG channel were used in whole-cell patch clamp electrophysiology studies at ambient temperature (26.5-28.5° C.). The methodology for stable transfection of this channel in HEK293 cells can be found elsewhere (Zhou et al 1998, Biophysical Journal, 74, pp 230-241). The solutions used for experimentation were standard extracellular solution of the following composition (mM); NaCl, 137; KCl, 4; CaCl₂, 1.8; MgCl₂, 1; Glucose, 10; HEPES, 10; pH 7.4±0.05 with NaOH/HCl; and standard intracellular solution of the following composition (mM); KCl 130; MgCl₂, 1; HEPES, 10; EGTA, 5; MgATP, 5; pH 7.2±0.05 with KOH. The voltage protocol applied was designed to activate the hERG channel and allow the measurement of drug block of the channel and is as follows. First the membrane potential was stepped from a holding potential of −80 mV to +30 mV for 1s. This was followed by a descending voltage ramp at a rate of 0.5 mV/ms back to holding potential of −80 mV and the peak outward current observed during the repolarizing ramp was measured. This protocol was evoked repeatedly every 4 seconds (0.25 Hz). After establishing a stable baseline period in the presence of vehicle (0.1% v/v DMSO), four increasing concentrations of test compound were then bath-applied sequentially until the response reached steady-state or 10 minutes (whichever occurred first). 10 micromol/L dofetilide was used at the end of each experiment as an internal positive control and to define maximum block. From this assay, the IC₅₀ of the compound was established.

### H3 Cell Based Functional Assay

The compound was evaluated using a cell based functional assay measuring cAMP through β-lactamase reporter gene activity. A stable cell line was generated from HEK-293 cells expressing a CRE β-lactamase reporter gene and transfected with human histamine H₃ receptor cDNA. Cells were seeded at a density of 500,000 cells/ml, and grown overnight in MEM (Invitrogen) supplemented with 1% dialysed FBS (Sigma), 2 mM glutamine (Sigma), 1 mM sodium pyruvate (Sigma), 0.1 mM non essential amino acids (Invitrogen) and 25 mM HEPES (Sigma) in poly D lysine coated 384 well plates (BD Biosciences). H₃ receptor agonist imetit (Tocris) dose dependently inhibited 10 μM forskolin (Calbiochem) stimulated synthesis of cAMP measured after 4.5 hours by β-lactamase cleavage of CCF4-AM dye (Invitrogen). For IC₅₀ determination, the test compound was prepared in PBS (Sigma) and DMSO (Sigma) at a dose response of 5×10⁻¹⁰ to 5×10⁻⁵ M with a final DMSO concentration in the assay of 0.5%. Cells were incubated for 15 minutes plus/minus compound and their ability to permit 10 μM forskolin-stimulated cAMP synthesis in the presence of 1 nM imetit was measured as described above. Functional Kᵢ values were calculated from the IC₅₀ of the compound tested as antagonists based on an experimentally determined imetit EC₅₀ (represented in the equation as K_{d}) of 350 pM, and an imetit concentration [L] of 1 nM, according to the Cheng-Prussoff equation where Kᵢ=(IC₅₀)/(1+([L]/K_{d})).

The compound of Example 1 was tested in the H₃ assay described above and found to have a Kᵢ value of 2.6 nM. The compound was also found to have an IC₅₀ value of 22200 nM in the hERG patch clamp assay.

1. The compound 6-[2-(1-Isopropyl-piperidin-4-yloxy)-7,8-dihydro-5H-[1,6]naphthyridin-6-yl]-nicotinamide

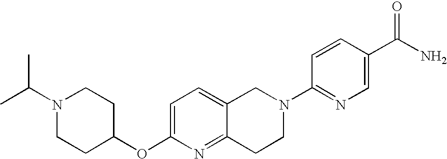

2. A pharmaceutical composition including the compound according to claim 1, together with a pharmaceutically acceptable excipient.

3. The compound according to claim 1, or a pharmaceutically acceptable salt or solvate thereof, for use as a medicament.

4. Use of a compound according to claim 1, or a pharmaceutically acceptable salt or solvate thereof, in the manufacture of a medicament to treat a disease for which a H₃ ligand is indicated.

5. The use according to claim 4, in the manufacture of a medicament for the treatment of sleep disorders, migraine, dyskinesia, stress-induced anxiety, psychotic disorders, epilepsy, Cognition deficiency diseases such as Alzheimer's disease or mild cognitive impairment, depression, mood disorders, schizophrenia, anxiety disorders, attention-deficit hyperactivity disorder (ADHD), psychotic disorders, eating disorders, weight control, obesity, dizziness, vertigo, epilepsy, motion sickness, female and male sexual dysfunction, inflammatory diseases, adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy-induced airway responses, allergic rhinitis, viral rhinitis, non-allergic rhinitis, perennial and seasonal rhinitis, nasal congestion and allergic congestion.

6. A method of treatment of a mammal, including a human being, suffering from a disease for which a H₃ ligand is indicated, comprising administering to said mammal an effective amount of the compound according to claim 1, or a pharmaceutically acceptable salt, solvate or composition thereof.

7. A combination of a compound according to claim 1 and another pharmacologically active agent.

8. The combination according to claim 7 wherein the other pharmalogically active agent is an histamine H₁ receptor antagonist.
